(19) United States
(12) Patent Application Publication
(10) Pub. No.: US 2020/0179548 A1
(43) Pub. Date: Jun. 11, 2020

(54) AUTOCLAVE OPERATION TO ACCOMMODATE FLUID STERILIZATION CYCLES

(71) Applicant: Immutrix Therapeutics, Inc., Rapid City, SD (US)

(72) Inventor: Sharif Anani, Rapid City, SD (US)

(21) Appl. No.: 16/705,870

(22) Filed: Dec. 6, 2019

Related U.S. Application Data
(60) Provisional application No. 62/776,846, filed on Dec. 7, 2018.

(51) Int. Cl. A61L 2/24; B01J 3/04; A61L 2/00; A61L 2/07

(52) U.S. Cl. CPC A61L 2/24; A61L 2/07; A61L 2/0094; B01J 3/04

(57) ABSTRACT

Embodiments relate generally to systems and methods for sterilizing a container comprising a liquid, where a method may comprise placing the container within an autoclave vessel; pressurizing the autoclave vessel to an initial pressure, by pressurizing the autoclave vessel with a clean gas via a first valve; after pressurizing the autoclave vessel to the initial pressure, heating the autoclave vessel to sterilize the liquid within the container within the autoclave vessel; controlling the pressure within the autoclave vessel while heating the autoclave vessel to maintain a pressure within ±10% of the initial pressure via a second valve; and cooling the autoclave vessel after sterilization.

### CROSS-REFERENCE TO RELATED APPLICATIONS

The present application claims priority to U.S. Provisional Patent Application Ser. No. 62/776,846 filed Dec. 7, 2018 by Sharif Anani and entitled “Autoclave Operation to Accommodate Fluid Sterilization Cycles” which is incorporated herein by reference as if reproduced in its entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Typical, conventional autoclave systems may include a pressure chamber used to carry out processes requiring temperatures and pressures elevated with respect to ambient air pressure and/or ambient temperature. In some cases, an autoclave may be configured to accommodate sterilization processes.

### SUMMARY

In an embodiment, a method for sterilizing a container comprising a liquid may comprise placing the container within an autoclave vessel; pressurizing the autoclave vessel to an initial pressure, by pressurizing the autoclave vessel with a clean gas via a first valve; after pressurizing the autoclave vessel to the initial pressure, heating the autoclave vessel to sterilize the liquid within the container within the autoclave vessel; controlling the pressure within the autoclave vessel while heating the autoclave vessel to maintain a pressure within ±10% of the initial pressure via a second valve; and cooling the autoclave vessel after sterilization.

In an embodiment, an autoclave system may comprise a sealable interior configured to receive an object to be sterilized; a source of clean gas configured to be fed to the sealable interior; a temperature controller configured to control the interior temperature of the sealable interior of the autoclave; and programmable logic controller, wherein the programmable logic controller and/or the temperature controller are configured to: pressurize the autoclave vessel to an initial pressure, by pressurizing the autoclave vessel with a clean gas via a first valve; after pressurizing the autoclave vessel to the initial pressure, heat the autoclave vessel to sterilize the liquid within the container within the autoclave vessel; control the pressure within the autoclave vessel while heating the autoclave vessel to maintain a pressure within ±10% of the initial pressure via a second valve; and cool the autoclave vessel after sterilization.

In an embodiment, a method of assembling a cartridge may comprise providing a cartridge body and at least one cap; disposing a fluid within the cartridge body; attaching the at least one cap to the cartridge body via an adhesive; placing the assembled cartridge containing the fluid within an autoclave; sealing the interior of the autoclave; increasing the pressure within the autoclave to a predetermined level (by pumping a clean gas into the interior of the autoclave, via a first valve); increasing the temperature within the autoclave to a predetermined level configured to sterilize the cartridge and/or fluid within the cartridge; maintaining the pressure at the predetermined level within the autoclave as the temperature is increased (by venting the interior of the autoclave, via a second valve); decreasing the temperature within the autoclave after a predetermine amount of time; decreasing the pressure within the autoclave; and providing the sterilized, assembled cartridge for use by a consumer.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates a method for assembling and/or producing a cartridge, according to an embodiment of the disclosure.

FIG. 2 illustrates an autoclave system according to an embodiment of the disclosure.

FIG. 3 illustrates a temperature-pressure profile for a typical autoclave system.

FIG. 4 illustrates a proposed temperature-pressure profile for an autoclave system according to an embodiment of the disclosure.

FIG. 5 illustrates an exemplary temperature-pressure profile for an autoclave system according to an embodiment of the disclosure.

FIG. 6 illustrates a block diagram of a computer system according to an embodiment of the disclosure.

Each figure shown in this disclosure shows a variation of an aspect of the embodiments presented, and only differences will be discussed in detail.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner in which it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present disclosure (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number, as understood by persons of skill in the art field (for example, ±10%); and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

In some embodiments of the disclosure are systems and methods for operating an autoclave system, and systems and methods for producing a cartridge that is sterilized using the disclosed autoclave systems and methods. Generally, a typical, conventional autoclave includes a pressure chamber used to carry out processes requiring temperatures and pressures elevated with respect to ambient air pressure. In some of the disclosed embodiments, the autoclave is configured to accommodate fluid sterilization cycles with the constraint of a rigid non-permeable container.

In some embodiments, the autoclave is configured for pre-pressurization, where the container housing the fluid is placed within the autoclave vessel, to prevent failure of the container housing the fluid that is to be sterilized. By pre-pressurizing the autoclave vessel to a pre-determined (or initial) pressure and venting during a subsequent heating process to stay within a threshold of the initial pressure, the autoclave is able to reach the required temperatures without damaging the container.

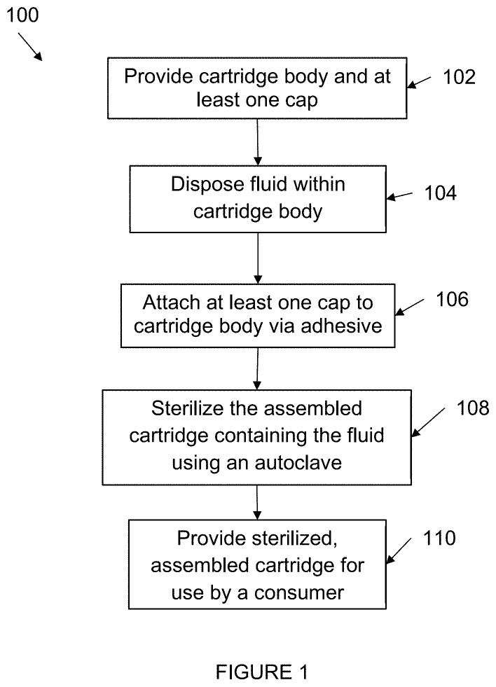

In some embodiments, the systems and methods for operating an autoclave system disclosed herein may be advantageously employed in the context of sterilization of a fluid-containing container or cartridge and/or to sterilize fluid within a container or cartridge, though the disclosed systems and methods may be similarly employed with respect to other sterilizations. For example, FIG. 1 illustrates an embodiment of a method 100 for producing (and/or assembling) a cartridge (or container) housing a fluid within the cartridge. In step 102, a cartridge may be provided, where the cartridge may generally comprise a cartridge body and at least one cap configured to be attached to the cartridge body. The process may comprise, at step 104, disposing a fluid within the cartridge body. The process may comprise, at step 106, attaching the at least one cap to the cartridge body containing the fluid, wherein the at least one cap may be attached via an adhesive or glue. The process may comprise, in step 108, sterilizing the assembled cartridge containing the fluid using an autoclave. Then the sterilized cartridge may be provided for use by a consumer, at step 110. It is to be understood the cartridge as described is an exemplary device and other devices are contemplated within this disclosure including for example blown glass containers or ampoules with or without an affixing mechanism. In an aspect, a device of this disclosure is utilized for the sterilization of any device having headspace wherein headspace refers to a portion of the device lacking a compressible medium (e.g., air).

In some embodiments, the process of sterilizing the assembled cartridge (for example, step 108) may comprise a sterilization process implemented via an autoclave.

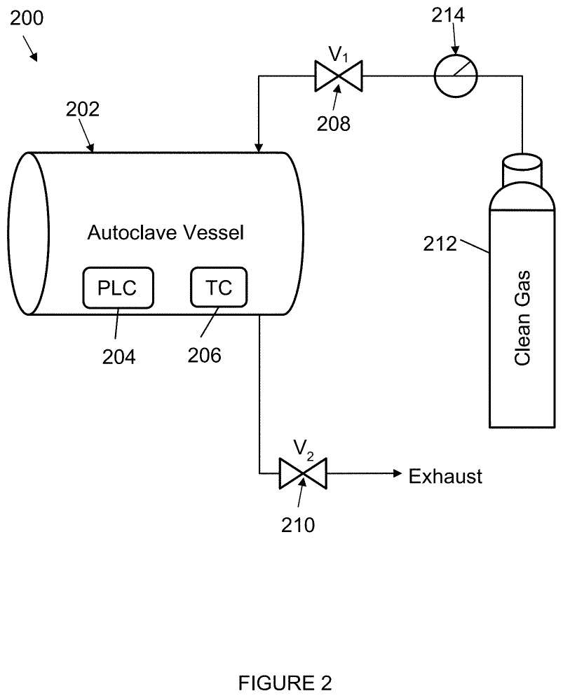

FIG. 2 illustrates aspects of an autoclave system 200 according to an embodiment of the disclosure. The autoclave system 200 may comprise an autoclave vessel 202 configured to receive an object to be sterilized (e.g., the container and/or cartridge). The autoclave system 200 may comprise an input valve 208 (e.g., a first valve) and an exhaust valve 210 (e.g., a second valve). The autoclave system 200 may also comprise a programmable logic controller (PLC) 204 (and/or any other programmable logic device) configured to communicate with and control one or more elements of the autoclave system 200. The autoclave system 200 may also comprise a temperature controller (TC) 206 configured to communicate with and control one or more elements of the autoclave system 200. In some embodiments, a single microcontroller may comprise the function of both the PLC 204 and the TC 206. In some embodiments, the PLC 204 and/or TC 206 may be configured to communicate with and control the input valve 208, the exhaust valve 210, or both the input valve 208 and the exhaust valve 210. Both the PLC and TC could be replaced with a single microcontroller board, but in some embodiments, keeping a divide between valve control (via the PLC) and temperature control (via the TC) may allow for easier debugging and quicker deployment.

In some embodiments the autoclave system 200 may comprise one or more sensors configured to detect/monitor temperature and/or pressure (e.g., within the autoclave vessel 202 and/or proximate to the autoclave vessel 202). For example, the PLC may receive signal from the one or more sensors, and then the PLC (and/or TC) may control a sterilization process using the received signals from the one or more sensors. In some embodiments, the autoclave system 200 may comprise a user interface configured to receive inputs from a user and/or display information to a user.

In some embodiments the sterilization process may comprise placing the cartridge within an autoclave vessel 202 and sealing the interior of the autoclave vessel 202. The sterilization process may comprise, prior to heating the interior space of the autoclave vessel 202, increasing (by the PLC 204) the pressure within the autoclave vessel 202 to a predetermined level (or predetermined pressure) (e.g., by pumping a clean gas 212 into the interior of the autoclave vessel 202, via a first valve 208). In some embodiments, the clean gas flow into the autoclave vessel 202 may be monitored and/or controlled by a regulator 214 in addition to the first (input) valve 208. The process may comprise increasing (by the TC) the temperature within the autoclave vessel 202 to a predetermined level (or predetermined temperature) configured to sterilize the cartridge and/or fluid within the cartridge. In some embodiments, the predetermined pressure and/or the predetermined temperature may be user chosen and/or user adjustable (for example, via a user interface). The process may comprise maintaining (by the PLC and/or TC) the pressure at (or near) the predetermined level within the autoclave vessel 202 as the temperature is increased (e.g., by venting the interior of the autoclave vessel 202, via a second valve 210). The process may comprise decreasing the temperature (by the PLC and/or TC) within the autoclave vessel 202 after a predetermined amount of time and decreasing (by the PLC) the pressure within the autoclave vessel 202.

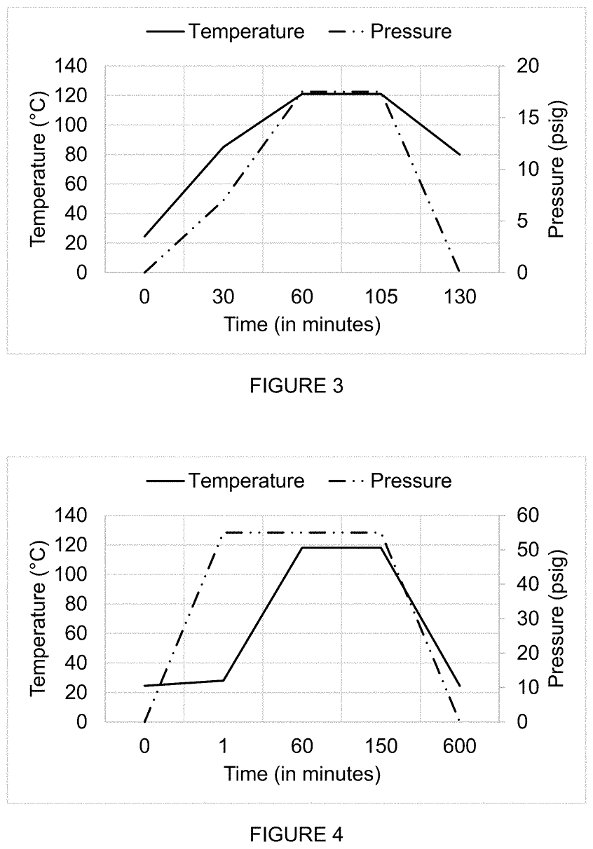

The autoclave system described herein may comprise advantages for sterilizing a container housing a liquid, as compared to typical autoclave systems that may not implement a pre-pressurization step, as shown in FIG. 3. A typical, conventional autoclave sterilization cycle uses heat to kill viable biological on a surface, and a typical autoclave system may use a valve to control venting of steam in order to create condensing steam for the purpose of transferring heat. However, a typical, conventional autoclave sterilization cycle may not be feasible when the sterilization subject is a fluid housed in a rigid, non-permeable container, due to the development of pressure inside the container when the fluid is heated to be sterilized. For example, the pressure inside the container may cause the container (e.g., the body of the container and/or any joints of the container that may be sealed with glue) to fail.

As shown in FIG. 3, a typical autoclave system may increase the temperature within the autoclave vessel, and then as the temperature is increased, the pressure may also increase. In the illustrated cycle in FIG. 3, pressure lags behind the temperature, where, as the temperature rises, water expands and evaporates, leading to higher pressures. In a system as illustrated by FIG. 3, the autoclave's computerized logic may actuate a venting valve that may typically aim to reach a pressure of somewhere between 15 and 17.5 psig (varies slightly with altitude).

To successfully sterilize fluids in a rigid, non-permeable container, the temperature and pressure profiles inside an autoclave may follow a temperature and pressure profile similar to that in FIG. 4. The system includes a step of pre-pressurizing the autoclave, which may reduce the pressure difference across the walls of the container at high temperatures, (e.g., when the container and/or adhesive attaching portions of the container is weakest). In other words, the exterior of the container (e.g., the autoclave vessel) may be pre-pressurized to a pressure approximate to the pressure expected within the interior of the container when the high temperatures are reached during the autoclave cycle. This may allow the pressure difference between the sides of the wall of the container to be lower when the temperature is higher.

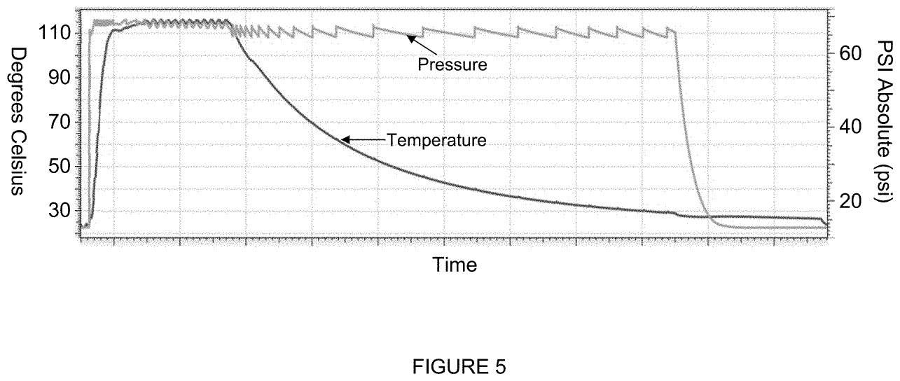

FIG. 5 illustrates an exemplary test result of an autoclave cycle that include a pre-pressurization step. At the beginning of the cycle, the pressure may be increased to approximately 68 psi (or between approximately 60 and 70 psi). Then, the temperature may be increased to at least approximately 110° C. (or between approximately 110° C. and 120° C.). In the example shown in FIG. 5, the temperature and pressure may be held within a range of approximately ±10% for approximately two hours. Then, to complete the cycle, the temperature may be gradually decreased over 7-8 hours, while the pressure may be held within a range of approximately ±10% for the majority of that time. Then, when the temperature has decreased, the pressure may be decreased (e.g., by venting the autoclave vessel) over approximately 30 minutes. The temperature may continue to decrease until the cycle in complete.

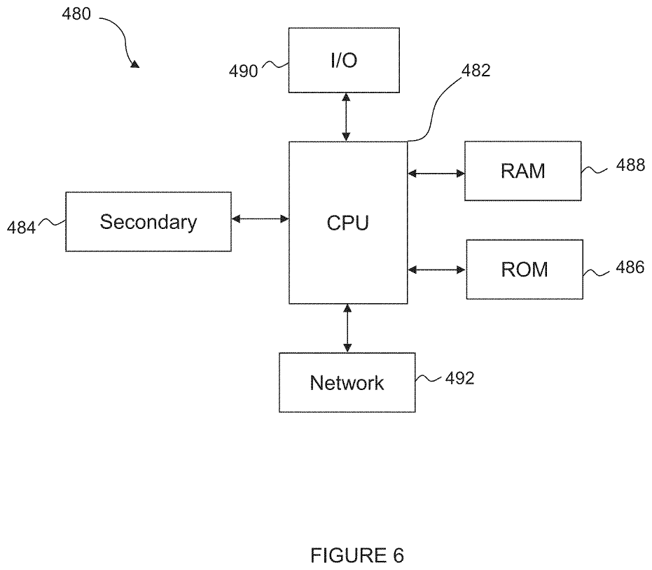

FIG. 6 illustrates a computer system 480 suitable for implementing one or more embodiments disclosed herein. For example, the system discussed above (e.g., the autoclave system comprising at least one of a PLC, TC, and/or microcontroller) may be implemented in a form substantially similar to that of the computer system 480 and the methods disclosed herein may be implemented by a computer system 480 programmed with executable instructions. Computer system 480 includes a processor 482 (which may be referred to as a central processor unit or CPU) that is in communication with memory devices including secondary storage 484, read only memory (ROM) 486, random access memory (RAM) 488, input/output (I/O) devices 490, and network connectivity devices 492. The processor 482 may be implemented as one or more CPU chips. The computer system 480 may comprise a power supply (not shown in FIG. 6) configured to power one or more of the elements of the computer system 480.

It is understood that by programming and/or loading executable instructions onto the computer system 480, at least one of the CPU 482, the RAM 488, and the ROM 486 are changed, transforming the computer system 480 in part into a particular machine or apparatus (e.g., a special purpose machine) having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

The secondary storage 484 is typically comprised of one or more disk drives or tape drives and is used for non-volatile storage of data and as an over-flow data storage device if RAM 488 is not large enough to hold all working data. Secondary storage 484 may be used to store programs which are loaded into RAM 488 when such programs are selected for execution. The ROM 486 is used to store instructions and perhaps data which are read during program execution. ROM 486 is a non-volatile memory device which typically has a small memory capacity relative to the larger memory capacity of secondary storage 484. The RAM 488 is used to store volatile data and perhaps to store instructions. Access to both ROM 486 and RAM 488 is typically faster than to secondary storage 484. The secondary storage 484, the RAM 488, and/or the ROM 486 may be referred to in some contexts as computer readable storage media and/or non-transitory computer readable media.

I/O devices 490 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 492 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. These network connectivity devices 492 may enable the processor 482 to communicate with the Internet or one or more intranets. With such a network connection, it is contemplated that the processor 482 might receive information from the network or might output information to the network in the course of performing the above-described method steps. Such information, which is often represented as a sequence of instructions to be executed using processor 482, may be received from and outputted to the network, for example, in the form of a computer data signal embodied in a carrier wave.

Such information, which may include data or instructions to be executed using processor 482 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well known to one skilled in the art. The baseband signal and/or signal embedded in the carrier wave may be referred to in some contexts as a transitory signal.

The processor 482 executes instructions, codes, computer programs, scripts which it accesses from hard disk, floppy disk, optical disk (these various disk-based systems may all be considered secondary storage 484), ROM 486, RAM 488, or the network connectivity devices 492. While only one processor 482 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 484, for example, hard drives, floppy disks, optical disks, and/or other device, the ROM 486, and/or the RAM 488 may be referred to in some contexts as non-transitory instructions and/or non-transitory information.

In an embodiment, the computer system 480 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 480 to provide the functionality of a number of servers that is not directly bound to the number of computers in the computer system 480. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

As various modifications could be made in the disclosed system, components, the methods of use herein described and illustrated without departing from the scope of the present disclosure, it is intended that all matter contained in the foregoing description or shown in the accompanying figures shall be interpreted as illustrative rather than limiting. Thus, the breadth and scope of the present disclosure should not be limited by any of the above described exemplary embodiments but should be defined only in accordance with the following claims appended hereto and their equivalents.

Having described various devices and methods herein, exemplary embodiments or aspects can include, but are not limited to:

In a first embodiment, a method for sterilizing a container comprising a liquid may comprise placing the container within an autoclave vessel; pressurizing the autoclave vessel to an initial pressure, by pressurizing the autoclave vessel with a clean gas via a first valve; after pressurizing the autoclave vessel to the initial pressure, heating the autoclave vessel to sterilize the liquid within the container within the autoclave vessel; controlling the pressure within the autoclave vessel while heating the autoclave vessel to maintain a pressure within ±10% of the initial pressure via a second valve; and cooling the autoclave vessel after sterilization.

A second embodiment can include the method of the first embodiment, wherein the container is assembled by a process that comprises: filling the container with the liquid; and sealing at least one cap onto a body of the container via an adhesive.

A third embodiment can include the method of the first or second embodiments, wherein the steps of pre-pressurizing the autoclave vessel and venting the autoclave vessel are controlled by a programmable logic controller in signal communication with the first valve and the second valve.

A fourth embodiment can include the method of any of the first to third embodiments, wherein the steps of heating the autoclave vessel and cooling the autoclave vessel are controlled by a temperature controller in signal communication with a temperature sensor.

In a fifth embodiment, an autoclave system may comprise a sealable interior configured to receive an object to be sterilized; a source of clean gas configured to be fed to the sealable interior; a temperature controller configured to control the interior temperature of the sealable interior of the autoclave; and programmable logic controller, wherein the programmable logic controller and/or the temperature controller are configured to: pressurize the autoclave vessel to an initial pressure, by pressurizing the autoclave vessel with a clean gas via a first valve; after pressurizing the autoclave vessel to the initial pressure, heat the autoclave vessel to sterilize the liquid within the container within the autoclave vessel; control the pressure within the autoclave vessel while heating the autoclave vessel to maintain a pressure within ±10% of the initial pressure via a second valve; and cool the autoclave vessel after sterilization.

A sixth embodiment can include the autoclave system of the fifth embodiment, wherein the object to be sterilized comprises a container housing a fluid.

A seventh embodiment can include the autoclave system of the fifth or sixth embodiment, wherein the container comprises a body and at least one cap, and wherein the at least one cap is attached to the body with an adhesive (or glue).

An eighth embodiment can include the autoclave system of the seventh embodiment, wherein the container body comprises a rigid material.

A ninth embodiment can include the autoclave system of the eighth embodiment, wherein the pre-determined pressure and the pre-determined temperature are chosen to prevent damage to the rigid material of the container body.

In a tenth embodiment, a method of assembling a cartridge may comprise providing a cartridge body and at least one cap; disposing a fluid within the cartridge body; attaching the at least one cap to the cartridge body via an adhesive; placing the assembled cartridge containing the fluid within an autoclave; sealing the interior of the autoclave; increasing the pressure within the autoclave to a predetermined level (by pumping a clean gas into the interior of the autoclave, via a first valve); increasing the temperature within the autoclave to a predetermined level configured to sterilize the cartridge and/or fluid within the cartridge; maintaining the pressure at the predetermined level within the autoclave as the temperature is increased (by venting the interior of the autoclave, via a second valve); decreasing the temperature within the autoclave after a predetermine amount of time; decreasing the pressure within the autoclave; and providing the sterilized, assembled cartridge for use by a consumer.

An eleventh embodiment can include the method of the tenth embodiment, wherein increasing the pressure within the autoclave and maintaining the pressure at the predetermined level are completed by a programmable logic controller of the autoclave.

A twelfth embodiment can include the method of the tenth or eleventh embodiments, wherein increasing the temperature within the autoclave and decreasing the temperature within the autoclave are completed by a temperature controller of the autoclave.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above but is defined by the claims which follow that scope including all equivalents of the subject matter of the claims. Each and every claim is incorporated as further disclosure into the specification and the claims are embodiment(s) of the present invention(s). Furthermore, any advantages and features described above may relate to specific embodiments but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically, and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method for sterilizing a container comprising a liquid, the method comprising:
placing the container within an autoclave vessel;
pressurizing the autoclave vessel to an initial pressure, by pressurizing the autoclave vessel with a clean gas via a first valve;
after pressurizing the autoclave vessel to the initial pressure, heating the autoclave vessel to sterilize the liquid within the container within the autoclave vessel;
controlling the pressure within the autoclave vessel while heating the autoclave vessel to maintain a pressure within ±10% of the initial pressure via a second valve; and
cooling the autoclave vessel after sterilization.

2. The method of claim 1, wherein the container is assembled by a process that comprises:
filling the container with the liquid; and
sealing at least one cap onto a body of the container via an adhesive.

3. The method of claim 1, wherein the steps of pre-pressurizing the autoclave vessel and venting the autoclave vessel are controlled by a programmable logic controller in signal communication with the first valve and the second valve.

4. The method of claim 1, wherein the steps of heating the autoclave vessel and cooling the autoclave vessel are controlled by a temperature controller in signal communication with a temperature sensor.

5. An autoclave system comprising:
a sealable interior configured to receive an object to be sterilized;
a source of clean gas configured to be fed to the sealable interior;
a temperature controller configured to control the interior temperature of the sealable interior of the autoclave; and
programmable logic controller, wherein the programmable logic controller and/or the temperature controller are configured to:
pressurize the autoclave vessel to an initial pressure, by pressurizing the autoclave vessel with a clean gas via a first valve;
after pressurizing the autoclave vessel to the initial pressure, heat the autoclave vessel to sterilize the liquid within the container within the autoclave vessel;
control the pressure within the autoclave vessel while heating the autoclave vessel to maintain a pressure within ±10% of the initial pressure via a second valve; and
cool the autoclave vessel after sterilization.

6. The autoclave system of claim 5, wherein the object to be sterilized comprises a container housing a fluid.

7. The autoclave system of claim 5, wherein the container comprises a body and at least one cap, and wherein the at least one cap is attached to the body with an adhesive (or glue).

8. The autoclave system of claim 7, wherein the container body comprises a rigid material.

9. The autoclave system of claim 8, wherein the pre-determined pressure and the pre-determined temperature are chosen to prevent damage to the rigid material of the container body.

10. A method of assembling a cartridge, the method comprising:
providing a cartridge body and at least one cap;
disposing a fluid within the cartridge body;
attaching the at least one cap to the cartridge body via an adhesive;
placing the assembled cartridge containing the fluid within an autoclave;
sealing the interior of the autoclave;
increasing the pressure within the autoclave to a predetermined level (by pumping a clean gas into the interior of the autoclave, via a first valve);
increasing the temperature within the autoclave to a predetermined level configured to sterilize the cartridge and/or fluid within the cartridge;
maintaining the pressure at the predetermined level within the autoclave as the temperature is increased (by venting the interior of the autoclave, via a second valve);
decreasing the temperature within the autoclave after a predetermine amount of time;
decreasing the pressure within the autoclave; and
providing the sterilized, assembled cartridge for use by a consumer.

11. The method of claim 10, wherein increasing the pressure within the autoclave and maintaining the pressure at the predetermined level are completed by a programmable logic controller of the autoclave.

12. The method of claim 10, wherein increasing the temperature within the autoclave and decreasing the temperature within the autoclave are completed by a temperature controller of the autoclave.
